(19) United States
(12) Patent Application Publication
(10) Pub. No.: US 2021/0244273 A1
(43) Pub. Date: Aug. 12, 2021

(54) EYE-TRACKING FUNDUS ILLUMINATION SYSTEM

(71) Applicant: Facebook Technologies, LLC, Menlo Park, CA (US)

(72) Inventors: Robin Sharma, Redmond, WA (US); Mohamed Tarek El-Haddad, Redmond, WA (US)

(21) Appl. No.: 17/078,745

(22) Filed: Oct. 23, 2020

Related U.S. Application Data
(60) Provisional application No. 62/970,945, filed on Feb. 6, 2020.

(51) Int. Cl. A61B 3/12; A61B 3/00; A61B 5/16

(52) U.S. Cl. CPC A61B 3/12; A61B 5/163; A61B 3/0008

(57) ABSTRACT

A fundus illumination system includes an eye-tracking system, an illumination source, a lens system, and a control module. The eye-tracking system is configured to track movements of an eye and the illumination source includes an array of light sources that selectively emit illumination light. The lens system is disposed between the illumination source and the eye to direct the illumination light to illuminate a fundus of the eye. The control module is communicatively coupled to the eye-tracking system and the illumination source, and selectively enables at least one light source of the array of light sources based on the movements of the eye to maintain illumination of the fundus.

### CROSS-REFERENCE TO RELATED APPLICATION

The present Application claims the benefit of U.S. Provisional Application No. 62/970,945, entitled “Eye-Tracking Fundus Illumination System” filed Feb. 6, 2020. U.S. Provisional Application No. 62/970,945 is expressly incorporated herein by reference in their entirety.

### FIELD OF DISCLOSURE

Aspects of the present disclosure relate generally to ocular fundus illumination and imaging systems.

### BACKGROUND

Fundus imaging involves imaging (e.g., photographing) the rear portion of the eye, also referred to as the fundus. In particular, the fundus of the eye is the interior surface of the eye, opposite the lens, and may include the retina, optic disc, macula, fovea, and posterior pole. In some contexts, analysis of fundus images may be useful by a care provider for diagnostic or treatment response purposes. For example, a physician may be able to identify issues, such as infections, degenerative eye diseases, or even congenital conditions based on the examination of the fundus images.

Some conventional fundus imaging systems may include various optics and a flash enabled camera. The operation of these conventional imaging systems may include directing a patient to fixate on a target image (e.g., a dot) that is projected onto the retina, and then flooding the pupil with light (e.g., activate the flash) to obtain the image.

### BRIEF DESCRIPTION OF THE DRAWINGS

Non-limiting and non-exhaustive aspects of the present disclosure are described with reference to the following figures, wherein like reference numerals refer to like parts throughout the various views unless otherwise specified.

FIGS. 1A and 1B illustrate a fundus imaging system, in accordance with aspects of the present disclosure.

FIGS. 2A, 2B, 3A, 3B, 4A, 4B, 5A, 5B, and 6, illustrate various eye movements and the corresponding translation of those movements to a position within an array of light sources, in accordance with aspects of the present disclosure.

FIG. 7 illustrates a computing device, in accordance with aspects of the present disclosure.

FIG. 8 is a flow chart illustrating a process of illuminating the fundus of an eye, in accordance with aspects of the present disclosure.

FIG. 9 illustrates a head mounted display (HMD), in accordance with aspects of the present disclosure.

### DETAILED DESCRIPTION

Various aspects and embodiments are disclosed in the following description and related drawings to show specific examples relating to a fundus illumination system that includes eye-tracking. Alternate aspects and embodiments will be apparent to those skilled in the pertinent art upon reading this disclosure and may be constructed and practiced without departing from the scope or spirit of the disclosure. Additionally, well-known elements will not be described in detail or may be omitted so as to not obscure the relevant details of the aspects and embodiments disclosed herein.

In some implementations of the disclosure, the term “near-eye” may be defined as including an element that is configured to be placed within 50 mm of an eye of a user while a near-eye device is being utilized. Therefore, a “near-eye optical element” or a “near-eye system” would include one or more elements configured to be placed within 50 mm of the eye of the user.

In aspects of this disclosure, visible light may be defined as having a wavelength range of approximately 380 nm-700 nm. Non-visible light may be defined as light having wavelengths that are outside the visible light range, such as ultraviolet light and infrared light. Infrared light having a wavelength range of approximately 700 nm-1 mm includes near-infrared light. In aspects of this disclosure, near-infrared light may be defined as having a wavelength range of approximately 700 nm-1.4 μm. White light may be defined as light that includes a broad range of wavelengths and in some instances may include multiple colors of the visible spectrum.

As mentioned above, the operation of some conventional fundus imaging systems may include directing a patient (or user) to fixate on a target image (e.g., a dot) that is projected onto their retina. One purpose of directing the patient to fixate on the target image is to ensure correct alignment between the eye and the illumination source (e.g. the flash) to maximize illumination of the fundus. However, in some instances, the patient may be unable to follow directions to fixate on the target due to a diminished capacity, either physical or developmental. For example, a user may have eye movement issues that prevents them from focusing on the target, or an infant/child may be unable to follow directions to focus on the target, and so on. If the eye is misaligned with the illumination source, the pupil of the eye may vignette the illumination light and prevent the light from reaching the fundus, which may degrade the resultant image.

Accordingly, aspects of the present disclosure provide a fundus illumination system that is invariant to eye movements and/or eye alignment. That is, a fundus illumination system may maintain illumination of the fundus even if the eye is not directly aligned with the illumination source and/or even as the eye moves. In one aspect, an illumination source is provided that includes an array of light sources. An eye tracker may also be provided that tracks movements of the eye, where one or more of the light sources in the array are selectively enabled to emit illumination light based on the determined movements of the eye to maintain the illumination of the fundus. These and other features will be described in more detail below.

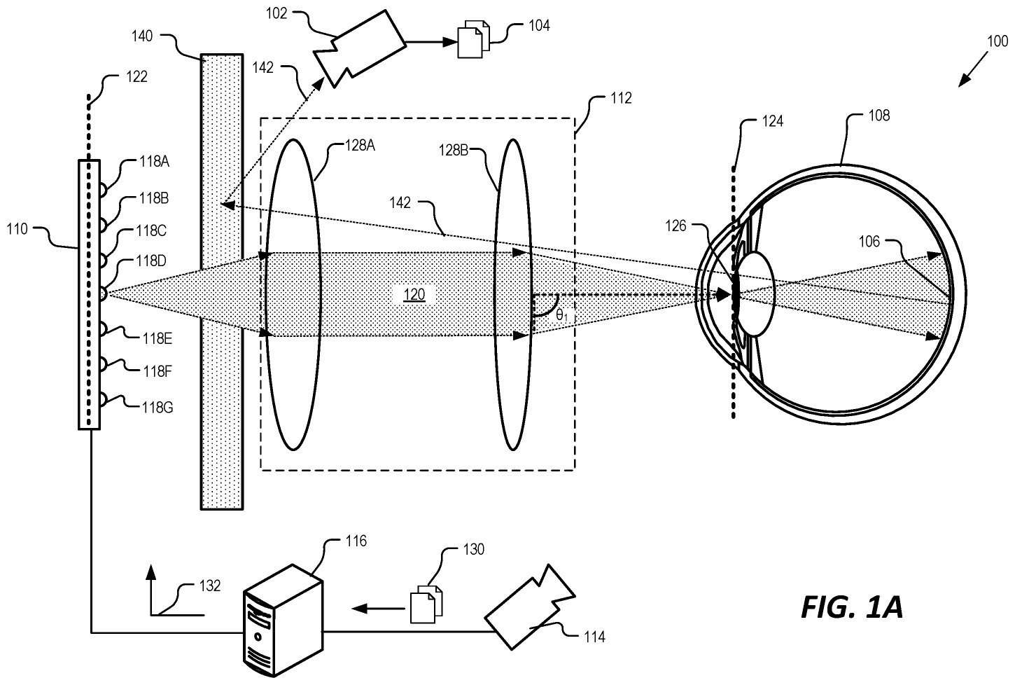

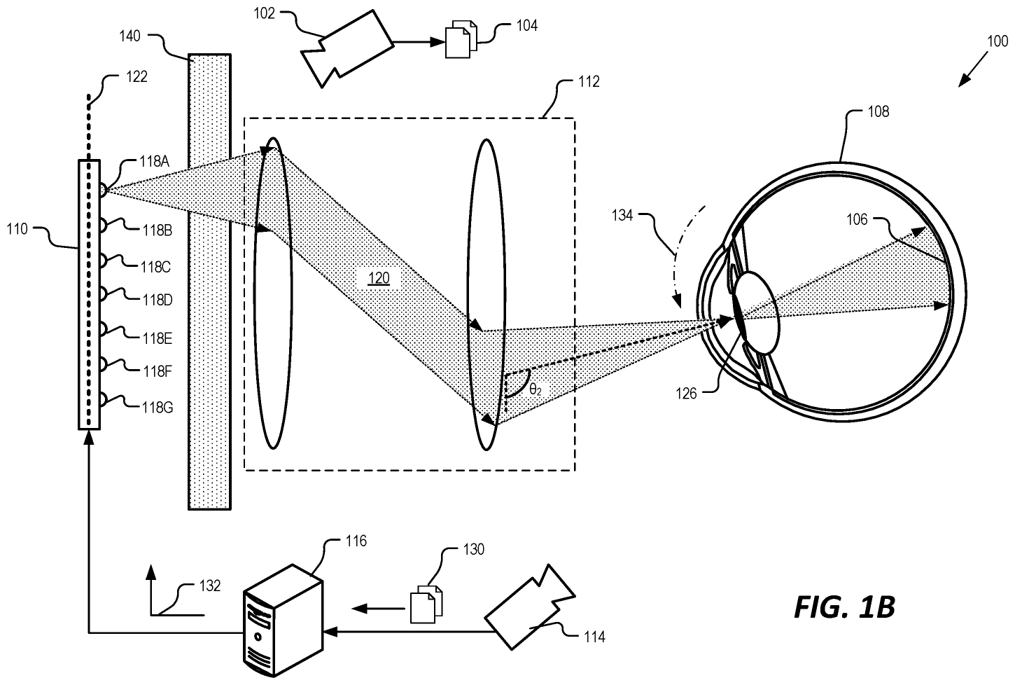

FIG. 1A illustrates a fundus imaging system 100, in accordance with aspects of the present disclosure. The illustrated example of the fundus imaging system 100 includes a first camera 102 that is configured to capture images 104 of a fundus 106 of eye 108. The fundus imaging system 100 is also shown as including an illumination source 110, a lens system 112, an eye-tracking system (e.g., a second camera 114), a combiner layer 140, and a computing device 116. As used herein, the illumination source 110, lens system 112, the eye-tracking system, the combiner layer 140, and the computing device 116 may be collectively referred to as a fundus illumination system.

In some examples, the illumination source 110 includes an array of light sources 118A-118G. Each light source 118A-118G may be a light-emitting diode (LED) that is configured to be selectively enabled to emit an illumination light 120. In one example, the illumination light 120 that is generated by the light sources 118A-118G is white light. In other examples, illumination light 120 is non-visible light, such as infrared light or near-infrared light. In some aspects, each light source 118A-118G is arranged within illumination source 110 in a two-dimensional (2D) array of columns and rows. In some examples, each light source 118A-118G may be referred to as a point light source, where only one of the light sources 118A-118G are enabled at a time to emit illumination light 120 (e.g., in the illustrated example of FIG. 1A, only a single light source 118D is currently enabled to emit illumination light 120).

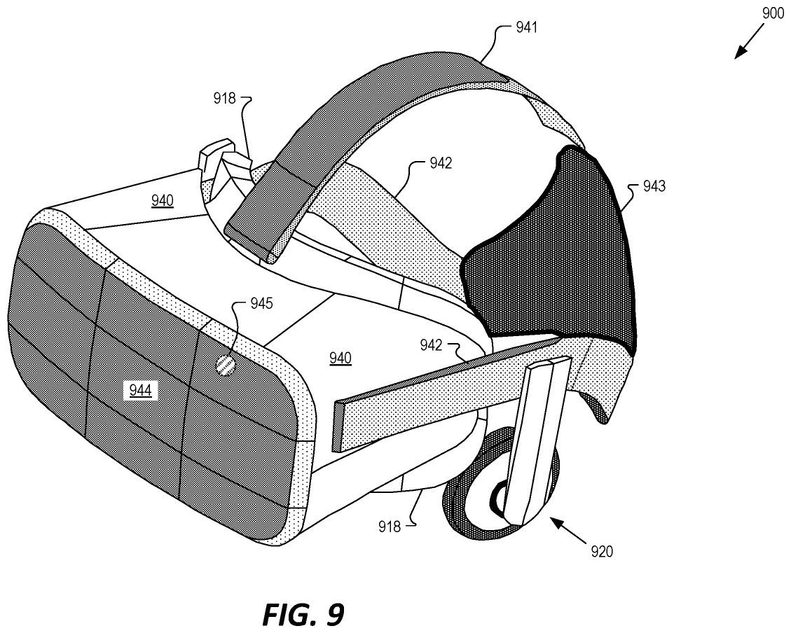

As shown in FIG. 1A, the illumination source 110 is positioned in a plane 122 that is conjugate to a pupil plane 124 of the eye 108. In some implementations, the positioning of the illumination source 110 with respect to the eye 108 is obtained by way of a head/chinrest stand (not shown) that is provided to the user/patient. In other implementations, the positioning is provided by way of a head-mounted device (e.g., see head-mounted display of FIG. 9).

As shown in FIG. 1A, the lens system 112 is configured to receive the illumination light 120 and direct the illumination light 120 to illuminate the fundus 106 of the eye 108. In some examples, the lens system 112 provides a Maxwellian view where the lens system 112 converges the illumination light 120 to a focal point at the pupil plane 124. As shown in FIG. 1A, the illumination light 120 then expands as it exits the pupil 126 towards to back of the eye 108 to illuminate a large area of the fundus 106. In some aspects, the lens system 112 includes a 4-F lens configuration of one or more Fourier transforming lenses (e.g., lenses 128A and 128B). Although the illustrated example of lens system 112 is shown as including two Fourier transforming lenses utilizing the transmission of illumination light 120, in other examples, the lens system 112 may alternatively include a single Fourier transforming lens that utilizes the reflection of the illumination light 120. In other examples, the lens system 112 may be omitted where a beam-steering system is incorporated to control the direction of the illumination light 120. By way of example, a beam-steering system may be liquid-crystal based, a scanning mirror, and/or a diffractive beam-steering system.

FIG. 1A illustrates a combiner layer 140 configured to receive reflected light 142 that is reflected off fundus 106 and redirect the reflected light 142 the camera 102, in accordance with an aspect of the disclosure. An optical combiner in combiner layer 140 is configured to be optically transparent to the illumination light 120 so that the illumination light 120 can propagate through combiner layer 140 to become incident on eye 108. In some embodiments, combiner layer 140 may be particularly configured to redirect the particular wavelength of light that is emitted by illumination source 110 to camera 102, even while generally passing other wavelengths of light.

As mentioned above, the fundus imaging system 100 may include an eye-tracking system to track movements of the eye 108. In the illustrated example, the eye-tracking system is provided by way of the second camera 114. The second camera 114 is configured to capture one or more images 130 of the eye 108. The second camera is communicatively coupled to computing device 116, which is configured to track movements of the eye 108 based on the one or more images 130. In some examples, the eye-tracking system is a pupil-tracker that is configured to determine the movements of the eye based on movements of the pupil 126.

In some examples, an eye-tracking module of the computing device 116 may be configured to determine eye-tracking information (e.g., location, orientation, gaze angle, etc. of the eye 108). In some aspects, the eye-tracking module may be configured to receive an image 130 captured by the second camera 114 and process the image to detect one or more specular reflections. The eye-tracking module may then localize the detected specular reflections to determine eye-tracking information (e.g., position, orientation, gaze angle, etc. of the eye 108). For example, the eye-tracking module may determine whether the eye 108 is looking in the straight, left, right, upwards, or downwards direction.

In some embodiments, the computing device 116 may include a control module that is communicatively coupled to the illumination source 110. As shown in FIG. 1A, the eye 108 is generally looking forward and is aligned with the illumination source 110. Thus, in this scenario a center light source (e.g., light source 118D) may be enabled to emit the illumination light 120. However, as mentioned above, if the eye 108 is not directly aligned, or if the eye 108 moves, the pupil 126 may vignette the illumination light 120. Accordingly, the control module of computing device 116 may generate one or more control signals 132 to selectively enable at least one of the light sources 118A-118G based on the detected movements of the eye 108 to maintain illumination of the fundus 106.

For example, in some aspects, each light source 118A-118G of the array of light sources may include a corresponding position within the array. The control module may be configured to translate the detected movements of the eye 108 to a position within the array to determine which of the light sources 118A-118G to enable. In some embodiments, changing which of the light sources 118A-118G is enabled changes an angle at which the illumination light 120 is emitted from the lens system 112. By way of example, FIG. 1A illustrates the illumination light 120 as being emitted from the lens system 112 at an angle θ₁ when light source 118D is enabled, whereas FIG. 1B illustrates the illumination light 120 as being emitted at an angle θ₂ when light source 118A is enabled. Thus, in operation, the eye-tracking module of computing device 116 may detect the movement 134 of eye 108 based on one or more of the images 130, where the control module then translates the detected movements 134 of the eye to a position of a light source in the array of light sources. As shown in FIG. 1B, the translation of the movement 134 to a position in the array results in the control module generating one or more control signals 132 to disable light sources 118D (as compared to that in FIG. 1A) and to enable light source 118A. The change from enabling light source 118D to enabling light source 118A changes the angle (e.g., θ₁ to θ₂) at which illumination light 120 is emitted from the lens system 112 to maintain illumination of the fundus 106.

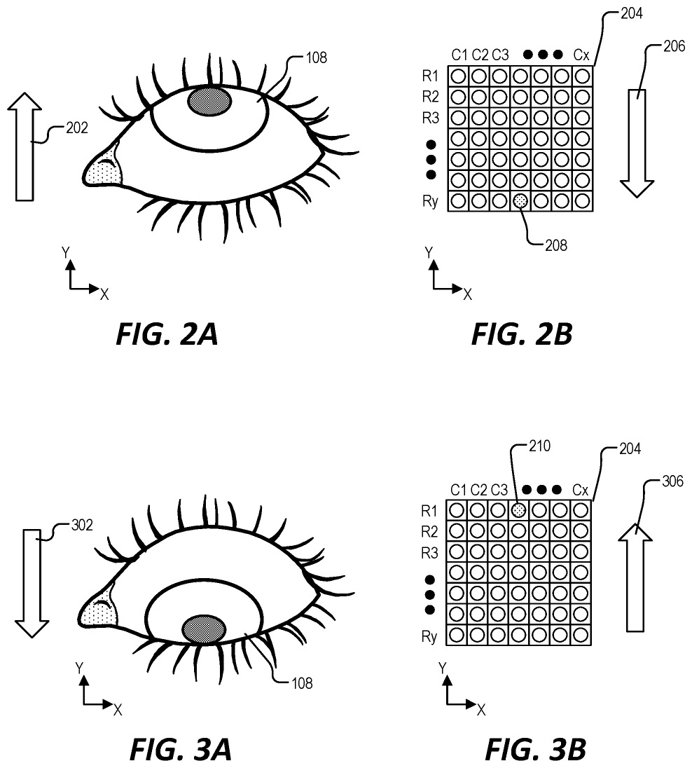

In some examples, the control module of computing device 116 is configured to translate the movements 134 of the eye 108 to a position within the array of light sources 118A-118G in a direction that is opposite to the movements 134. By way of example, FIG. 2A illustrates an eye movement that is in an upwards direction 202. FIG. 2B illustrates an eye-facing side of an array of light sources 204 that may be included in a illumination source, such as illumination source 110 of FIGS. 1A and 1B. As shown in FIG. 2B, the control module has translated the upwards movement of the eye 108 to a position within the array of light sources 204 that is in a downwards direction 206 (i.e., opposite the upwards direction 202 of the eye movements), such that light source 208 is now enabled to emit illumination light. Similarly, FIG. 3A illustrates an eye movement that is in a downwards direction 302, whereas FIG. 3B illustrates the translation of the downwards movement of the eye 108 to a position within the array of light sources 204 that is in an upwards direction 306, such that light source 210 is enabled.

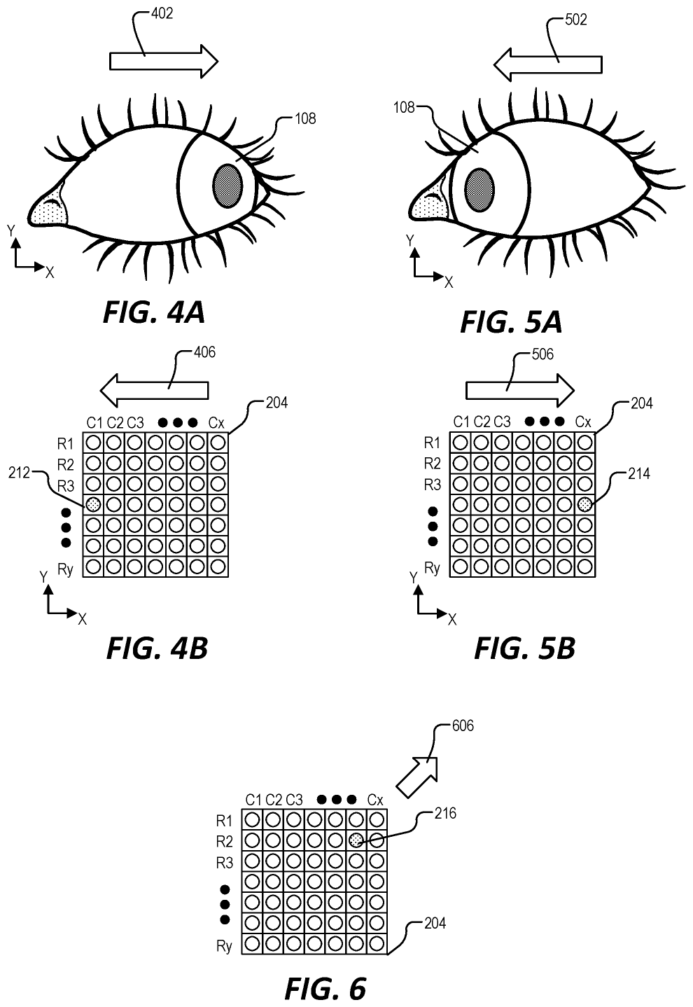

In another example, FIG. 4A illustrates an eye movement that is in an outward or temple direction 402 (i.e., towards the user's temple), whereas FIG. 4B illustrates the translation of that outward movement of the eye 108 to a position within the array of light sources 204 that is in an inwards or nasal direction 406 (i.e., towards the user's nose), such that light source 212 is enabled. In another example, FIG. 5A illustrates an eye movement that is in the inward/nasal direction 502, whereas FIG. 5B illustrates the translation of that inward/nasal movement of the eye 108 to a position within the array of light sources 204 that is in outwards/temple direction 506, such that light source 214 is enabled.

The above examples of FIGS. 2A-5B illustrate the translation of eye movements along the x or the y axes. However, the control module of the computing device 116 may also be configured to translate eye movements that occur along both the x and y axes. By way of example, FIG. 6 illustrates the translation of a down-left movement of the eye to a position within the array of light sources 204 that is in an upwards-right direction 606, such that light source 216 is enabled. In addition to direction, the control module is configured to translate the magnitude of eye movements (i.e., larger eye movements are translated to larger position changes within the array of light sources, etc.).

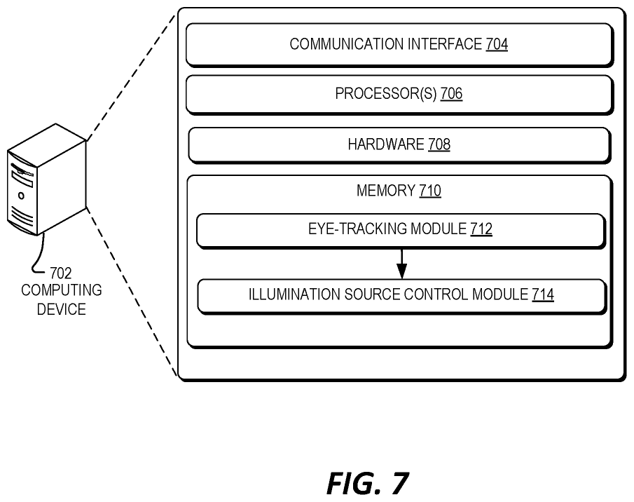

FIG. 7 illustrates a computing device 702, in accordance with aspects of the present disclosure. The illustrated example of computing device 702 is shown as including a communication interface 704, one or more processors 706, hardware 708, and a memory 710. The computing device 702 of FIG. 7 is one possible implementation of the computing device 116 of FIG. 1A.

The communication interface 704 may include wireless and/or wired communication components that enable the computing device 702 to transmit data to and receive data from other devices/components. The hardware 708 may include additional hardware interface, data communication, or data storage hardware. For example, the hardware interfaces may include a data output device, and one or more data input devices.

The memory 710 may be implemented using computer-readable media, such as computer storage media. In some aspects, computer-readable media may include volatile and/or non-volatile, removable and/or non-removable media implemented in any method or technology for storage of information such as computer-readable instructions, data structures, program modules, or other data. Computer-readable media includes, but is not limited to, RAM, ROM, EEPROM, flash memory or other memory technology, CD-ROM, digital versatile disks (DVD), high-definition multimedia/data storage disks, or other optical storage, magnetic cassettes, magnetic tape, magnetic disk storage or other magnetic storage devices, or any other non-transmission medium that can be used to store information for access by a computing device.

The processors 706 and the memory 710 of the computing device 702 may implement an eye-tracking module 712 and a illumination source control module 714. The eye-tracking module 712 and the illumination source control module 714 may include routines, program instructions, objects, and/or data structures that perform particular tasks or implement particular abstract data types. The memory 710 may also include a data store (not shown) that is used by the eye-tracking module 712 and/or illumination source control module 714.

The eye-tracking module 712 may be configured to receive images (e.g., images 130 of FIG. 1A) and process the images to determine a position and/or movements of the eye 108. The eye-tracking module 712 may then communicate with the illumination source control module 714 based on the determined movements/position. The illumination source control module 714 may be configured to translate the eye movements to a position within the array of light sources of the illumination source (e.g., illumination source 110 of FIG. 1A) and generate one or more control signals (e.g., control signals 132) to enable at least one of the light sources 118A-118G to maintain illumination of the fundus 106.

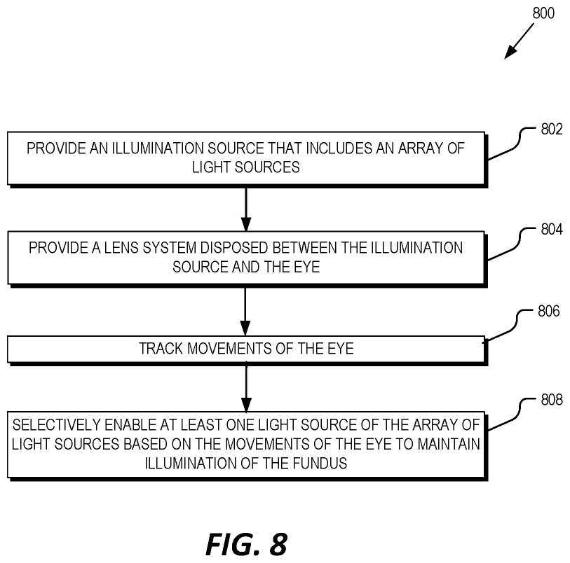

FIG. 8 is a flow chart illustrating a process 800 of illuminating the fundus of an eye, in accordance with aspects of the present disclosure. Process 800 includes one or more process blocks that may be performed by the computing device 116 of FIG. 1A and/or the computing device 702 of FIG. 7.

Process block 802 includes providing a illumination source that includes an array of light sources (e.g., illumination source 110 of FIG. 1A). Process block 804 includes providing a lens system (e.g., lens system 112 of FIG. 1A) between the illumination source and an eye (e.g., eye 108 of FIG. 1A). In some implementations, providing the illumination source and the lens system of process blocks 802 and 804 includes positioning the fundus imaging system 100 near the eye 108.

In a process block 806, movements of the eye 108 are tracked. As discussed above, an eye-tracking system, such as the second camera 114 of FIG. 1A may capture one or more images 130 of the eye, where the computing device 116 then analyzes the images to determine the position and/or movements of the eye 108. Next in a process block 808, the computing device 116 selectively enables at least one light source of the array of light sources based on the movements of the eye. As discussed above, the enabling of a light source may include translating the movements of the eye 108 to a position within the array of light sources to maintain illumination of the fundus 106 with illumination light 120.

In some implementations, aspects of the present disclosure, such as fundus imaging system 100 of FIG. 1A, may be utilized in a health care context for diagnostic or treatment response purposes. In such an implementation, one or more of the light sources (e.g., light sources 118A-118G) may be configured to emit white light for generating color images of the fundus. Having color images of the fundus may aid a care provider in ascertaining the health of the eye.

In other implementations, aspects of the present disclosure may be utilized in a head mounted device, such as a virtual reality (VR) or augmented reality (AR) device. In some aspects, a head mounted device may incorporate an eye-tracking system to enhance a user's viewing experience. Eye-tracking, may in some instances, be aided by determining the position and/or movement of one or more features present in the fundus of the eye. For example, a head mounted device may be configured to identify a fovea region from an image of the fundus and then determine a gaze angle of the eye based on the identified fovea region. The fovea region may be determined using one or more image processing techniques. When the gaze angle is determined, a virtual image presented to a user by a display of a head mounted device may be adjusted in response to the determined gaze angle.

By way of example, FIG. 9 illustrates a head-mounted display (HMD) 900, in accordance with aspects of the present disclosure. An HMD, such as HMD 900, is one type of head mounted device, typically worn on the head of a user to provide artificial reality content to a user. Artificial reality is a form of reality that has been adjusted in some manner before presentation to the user, which may include, e.g., virtual reality (VR), augmented reality (AR), mixed reality (MR), hybrid reality, or some combination and/or derivative thereof. The illustrated example of HMD 900 is shown as including a viewing structure 940, a top securing structure 941, a side securing structure 942, a rear securing structure 943, and a front rigid body 944. In some examples, the HMD 900 is configured to be worn on a head of a user of the HMD 900, where the top securing structure 941, side securing structure 942, and/or rear securing structure 943 may include a fabric strap including elastic as well as one or more rigid structures (e.g., plastic) for securing the HMD 900 to the head of the user. HMD 900 may also optionally include one or more earpieces 920 for delivering audio to the ear(s) of the user of the HMD 900.

The illustrated example of HMD 900 also includes an interface membrane 918 for contacting a face of the user of the HMD 900, where the interface membrane 918 functions to block out at least some ambient light from reaching to the eyes of the user of the HMD 900.

Example HMD 900 may also include a chassis for supporting hardware of the viewing structure 940 of HMD 900 (chassis and hardware not explicitly illustrated in FIG. 9). The hardware of viewing structure 940 may include any of processing logic, wired and/or wireless data interface for sending and receiving data, graphic processors, and one or more memories for storing data and computer-executable instructions. In one example, viewing structure 940 may be configured to receive wired power and/or may be configured to be powered by one or more batteries. In addition, viewing structure 940 may be configured to receive wired and/or wireless data including video data.

Viewing structure 940 may include a display system having one or more electronic displays for directing light to the eye(s) of a user of HMD 900. The display system may include one or more of a liquid crystal display (LCD), an organic light emitting diode (OLED) display, a micro-LED display, etc. for emitting light (e.g., content, images, video, etc.) to a user of HMD 900. The viewing structure 940 may also include an optical assembly that is configured to receive the image light from the display system and generate a virtual image (e.g., by collimating the image light) for viewing by an eye of a wearer of the HMD 900.

In some examples, viewing structure includes a fundus imaging system 945 for obtaining one or more images of a fundus of the user's eye. The fundus imaging system 945 may be implemented by way of any of the embodiments discussed herein, including fundus imaging system 100 of FIG. 1A. In some examples, when fundus imaging system 100 is incorporated into an HMD, such as HMD 900, the light sources 118A-118G may be configured to emit non-visible illumination light (e.g., IR, NIR, etc.), so as to not interfere with the user's viewing experience of the display.

Embodiments of the invention may include or be implemented in conjunction with an artificial reality system. Artificial reality is a form of reality that has been adjusted in some manner before presentation to a user, which may include, e.g., a virtual reality (VR), an augmented reality (AR), a mixed reality (MR), a hybrid reality, or some combination and/or derivatives thereof. Artificial reality content may include completely generated content or generated content combined with captured (e.g., real-world) content. The artificial reality content may include video, audio, haptic feedback, or some combination thereof, and any of which may be presented in a single channel or in multiple channels (such as stereo video that produces a three-dimensional effect to the viewer). Additionally, in some embodiments, artificial reality may also be associated with applications, products, accessories, services, or some combination thereof, that are used to, e.g., create content in an artificial reality and/or are otherwise used in (e.g., perform activities in) an artificial reality. The artificial reality system that provides the artificial reality content may be implemented on various platforms, including a head-mounted display (HMD) connected to a host computer system, a standalone HMD, a mobile device or computing system, or any other hardware platform capable of providing artificial reality content to one or more viewers.

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the following claims should not be construed to limit the invention to the specific embodiments disclosed in the specification. Rather, the scope of the invention is to be determined entirely by the following claims, which are to be construed in accordance with established doctrines of claim interpretation.

What is claimed is:

1. A fundus illumination system, comprising:
an eye-tracking system configured to track movements of an eye;
an illumination source that includes an array of light sources configured to be selectively enabled to emit illumination light;
a lens system disposed between the illumination source and the eye to direct the illumination light generated by the illumination source to illuminate a fundus of the eye; and
a control module communicatively coupled to the eye-tracking system and the illumination source, wherein the control module is configured to selectively enable at least one light source of the array of light sources based on the movements of the eye to maintain illumination of the fundus.

2. The fundus illumination system of claim 1, wherein the eye-tracking system comprises a camera to capture at least one image of the eye and is configured to determine the movements of the eye based on the at least one image.

3. The fundus illumination system of claim 1, wherein the eye-tracking system comprises a pupil-tracker to determine the movements of the eye based on movements of a pupil of the eye.

4. The fundus illumination system of claim 1, wherein the illumination source is configured to be placed in a plane that is conjugate to a pupil plane of the eye.

5. The fundus illumination system of claim 1, wherein the illumination light is non-visible light, infrared light, or near-infrared light.

6. The fundus illumination system of claim 1, wherein the array of light sources comprises an array of light emitting diodes (LEDs).

7. The fundus illumination system of claim 1, wherein the lens system comprises a 4-F lens configuration of one or more Fourier transforming lenses.

8. The fundus illumination system of claim 1, wherein each light source of the array of light sources includes a corresponding position within the array of light sources, and wherein the control module is configured to translate the movements of the eye to a position within the array of light sources to determine which of the at least one light sources to enable.

9. The fundus illumination system of claim 8, wherein the control module is configured to translate the movements of the eye to the position within the array of light sources in a direction that is opposite to the movements of the eye.

10. The fundus illumination system of claim 8, wherein a change from enabling a first light source at a first position within the array of light sources to enabling a second light source at a second position within the array of light sources changes an angle at which the illumination light is emitted from the lens system to maintain the illumination of the fundus.

11. A method of illuminating a fundus of an eye, the method comprising:
providing an illumination source that includes an array of light sources configured to be selectively enabled to emit illumination light;
providing a lens system disposed between the illumination source and the eye to direct the illumination light generated by the illumination source to illuminate the fundus of the eye;
tracking movements of the eye; and
selectively enabling at least one light source of the array of light sources based on the movements of the eye to maintain illumination of the fundus.

12. The method of claim 11, wherein tracking the movements of the eye, comprises:
capturing at least one image of the eye; and
determining the movements of the eye based on the at least one image.

13. The method of claim 11, wherein tracking the movements of the eye comprises tracking movements of a pupil of the eye.

14. The method of claim 11, wherein the illumination light is non-visible light, infrared light, or near-infrared light.

15. The method of claim 11, wherein each light source of the array of light sources includes a corresponding position within the array of light sources, and wherein selectively enabling the at least one light source comprises:
translating the movements of the eye to a position within the array of light sources to determine which of the at least one light sources to enable.

16. The method of claim 15, wherein translating the movements of the eye comprises:
translating the movements of the eye to the position within the array of light sources in a direction that is opposite to the movements of the eye.

17. The method of claim 15, wherein a change from enabling a first light source at a first position within the array of light sources to enabling a second light source at a second position within the array of light sources changes an angle at which the illumination light is emitted from the lens system to maintain the illumination of the fundus.

18. A fundus imaging system, comprising:
a first camera configured to capture at least one image of an eye;
an eye-tracking module coupled to the first camera to determine movements of the eye based on the at least one image;
an illumination source that includes an array of light sources configured to be selectively enabled to emit illumination light, wherein the illumination source is configured to be placed in a plane that is conjugate to a pupil plane of the eye;
a lens system disposed between the illumination source and the eye to direct the illumination light generated by the illumination source to illuminate a fundus of the eye;
a control module configured to selectively enable at least one light source of the array of light sources based on the movements of the eye to maintain illumination of the fundus; and
a second camera configured to capture at least one image of the fundus while the fundus is illuminated with the illumination light.

19. The fundus imaging system of claim 18, wherein each light source of the array of light sources includes a corresponding position within the array of light sources, and wherein the control module is configured to translate the movements of the eye to a position within the array of light sources to determine which of the at least one light sources to enable.

20. The fundus imaging system of claim 19, wherein a change from enabling a first light source at a first position within the array of light sources to enabling a second light source at a second position within the array of light sources changes an angle at which the illumination light is emitted from the lens system to maintain the illumination of the fundus.
